(19) United States
(12) Patent Application Publication
(10) Pub. No.: US 2026/0213002 A1
(43) Pub. Date: Jul. 23, 2026

(54) PREDICTIVE AUTOMATED MEDICAL EXAMINATION RESOURCE MANAGEMENT SYSTEM

(71) Applicant: SAMSUNG LIFE PUBLIC WELFARE FOUNDATION, Seoul (KR)

(72) Inventors: Junghwan MOON, Seoul (KR); Keunhwa KIM, Seoul (KR); Choonbong SON, Seoul (KR); Byoungjun HWANG, Seoul (KR)

(73) Assignee: SAMSUNG LIFE PUBLIC WELFARE FOUNDATION, Seoul (KR)

(21) Appl. No.: 19/446,721

(22) Filed: Jan. 12, 2026

Related U.S. Application Data
(63) Continuation-in-part of application No. 18/060,039, filed on Nov. 30, 2022, now abandoned.

(30) Foreign Application Priority Data
Dec. 1, 2021 (KR) 10-2021-0170235

(51) Int. Cl. G16H 40/20; G16H 30/20; G16H 50/70

(52) U.S. Cl. CPC G16H 40/20; G16H 30/20; G16H 50/70

(57) ABSTRACT

A system for performing prediction-based automated medical examination resource management, the system being configured to collect raw data related to computerized tomography (CT) examinations from an electronic medical record (EMR) system or a hospital information system (HIS), predict a predicted demand curve representing a trend of a future average number of daily prescriptions based on the raw data by using machine learning, derive a predicted waiting day curve indicating expected waiting days for each future date by using the predicted demand curve and available examination capacity (CAPA) data of a CT scanner equipment for each date, and automatically transmit a control signal to a hospital scheduling system or CT scanner equipment when the expected waiting date for each future date on the predicted waiting day curve satisfies a predefined threshold condition.

### CROSS-REFERENCE TO RELATED APPLICATION(S)

This application is a continuation-in-part of and claims benefit under 35 U.S.C. § 120 to U.S. application Ser. No. 18/060,039, filed Nov. 30, 2022, which is based on and claims priority under 35 U.S.C. § 119 to Korean Patent Application No. 10-2021-0170235, filed on Dec. 1, 2021, in the Korean Intellectual Property Office, the entire contents of each of which are incorporated herein by reference.

### BACKGROUND

### 1. Field

One or more embodiments relate to a predictive automated medical examination resource management system.

### 2. Description of the Related Art

In line with the improved quality of life and the increased interest in health, the population using medical services is gradually increasing, and there has been a request for efficient management of reservations of medical services such as diagnosis, treatment, operations conducted by clinics and hospitals. To minimize the waiting from the standpoint of individuals, and for efficient resource management on the side of clinics and hospitals, it has now become common to manage the examination schedule through reservations at clinics and hospitals.

However, at the current point of reservation, only days to wait for until the examination is provided, but the expected waiting days at the future time point is not provided, and thus, it is difficult for individuals to manage the schedule, and at the end of the hospitals and clinics, it is difficult to set up suitable investment and operation plan for the examination equipment in advance, which delays the examinations.

In large hospital settings, expensive diagnostic equipment such as computed tomography (CT) scanners play a crucial role in patient care, but they also pose significant operational and financial challenges. Demand for these devices continues to grow, while resources are limited, resulting in chronically long appointment waiting days. Existing hospital information systems only provide current availability and current waiting days. This fundamentally hinders hospital managers from predicting future demand changes and proactively allocating resources based on these changes. Consequently, in the short term, this leads to delays in patient care and reduced satisfaction. In the medium to long term, this leads to inefficient equipment operation, unnecessary overtime, and a lack of data-driven support for critical capital investment decisions, such as new equipment acquisition or staffing.

### SUMMARY

One or more embodiments include a predictive automated medical examination resource management system whereby waiting days for an examination that requires a reservation at a hospital may be efficiently predicted. Also, one or more embodiments include a transition of a hospital operation paradigm via predictive automated resource management. However, the above objectives of the disclosure are exemplary, and the scope of the disclosure is not limited by the above objectives.

Additional aspects will be set forth in part in the description which follows and, in part, will be apparent from the description, or may be learned by practice of the presented embodiments of the disclosure.

According to one or more embodiments, a method of predicting waiting days for a medical examination requiring a reservation at a hospital, includes acquiring examination prescription (where a ‘prescription’ means an effective prescription, for which a reservation is made or an execution is conducted after the prescription, except for repeated prescriptions) and execution data, calculating, based on the examination prescription and execution data, an average ratio of executed examinations for each day required from a prescription to the execution thereof, and an average number of prescriptions based on past weekdays (where a ‘weekday’ means a working day), estimating, based on the average number of prescriptions based on past weekdays, an average number of prescriptions based on future weekdays for a predetermined period by using at least one time-series prediction model, calculating a monthly increment in the average number of prescriptions based on future weekdays, compared to the average number of prescriptions based on the past weekdays, and allocating, based on the average number of prescriptions based on the past weekdays and the monthly increment, an expected number of prescriptions based on future weekdays, and estimating waiting days for an examination for each future date based on the expected number of prescriptions,

The acquiring of the examination prescription and execution data may include distinguishing prescription departments by reflecting a difference in examination prescription and execution patterns between weekdays and weekends, and acquiring prescription data of prescriptions of the prescription departments for a predetermined period, prescription data of prescriptions, for which examinations are conducted after the prescriptions, and execution data.

The calculating of the average ratio of executed examinations for each day required from a prescription to the execution thereof, and the average number of prescriptions based on past weekdays may include calculating a number of prescriptions by date with respect to each prescription department based on the execution data, calculating an average ratio of executed examinations for each day required from a prescription to the execution thereof, based on the prescription data and the number of prescriptions by date, and calculating the average number of prescriptions based on past weekdays, by dividing the number of prescriptions for a predetermined period, by the number of weekdays

The estimating of the average number of prescriptions based on future weekdays may include estimating the average number of prescriptions based on future weekdays by using an ensemble model which combines result values of a plurality of time-series prediction models.

The estimating of waiting days for an examination for each future date may include setting an additional capacity that is additionally operable in addition to the capacity, when a date of examination requiring a reservation is a weekday, and when the estimated number of reservations for the date of examination exceeds a sum of the capacity and the additional capacity, distributing the excess capacity to a date prior to the date of examination according to a predetermined condition.

The estimating of the waiting days for an examination for each future date may include calculating, as waiting days for each future date, a section until a first day when two consecutive weekdays start to appear, on which the number of reservations compared to the capacity for each future date is less than or equal to a predetermined ratio.

According to one or more embodiment, a predictive automated medical examination resource management system includes a processor configured to acquire examination prescription and execution data, calculate, based on the examination prescription and execution data, an average ratio of executed examinations for each day required from a prescription to the execution thereof, and an average number of prescriptions based on past weekdays, estimate, based on the average number of prescriptions based on past weekdays, an average number of prescriptions based on future weekdays for a predetermined period by using at least one time-series prediction model, calculate a monthly increment in the average number of prescriptions based on future weekdays, compared to the average number of prescriptions based on the past weekdays, and allocate, based on the average number of prescriptions based on the past weekdays and the monthly increment, an expected number of prescriptions based on future weekdays, and estimate waiting days for an examination for each future date based on the expected number of prescriptions.

The processor may be further configured to distinguish prescription departments by reflecting a difference in examination prescription and execution patterns between weekdays and weekends, and acquire prescription data of prescriptions of the prescription departments for a predetermined period, prescription data of prescriptions, for which examinations are conducted after the prescriptions, and execution data.

The processor may be further configured to calculate a number of prescriptions by date with respect to each prescription department based on the execution data, calculate an average ratio of executed examinations for each day required from a prescription to the execution thereof, based on the prescription data and the number of prescriptions by date, and calculate the average number of prescriptions based on past weekdays, by dividing the number of prescriptions for a predetermined period, by the number of weekdays.

The processor may be further configured to estimate the average number of prescriptions based on future weekdays by using an ensemble model which combines result values of a plurality of time-series prediction models.

The processor may be further configured to set an additional capacity that is additionally operable in addition to the capacity, when a date of examination requiring a reservation is a weekday, and when the estimated number of reservations for the date of examination exceeds a sum of the capacity and the additional capacity, to distribute the excess capacity to a date prior to the date of examination according to a predetermined condition.

The processor may be further configured to calculate, as waiting days for each future date, a section until a first day when two consecutive weekdays start to appear, on which the number of reservations compared to the capacity for each future date is less than or equal to a predetermined ratio.

According to one or more embodiments, a computer program stored on a recording medium for executing the method above by using a computer is provided.

In addition to the aforesaid details, other aspects, features, and advantages will be clarified from the following drawings, claims, and detailed description.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram for describing the structure and operation of a predictive automated medical examination resource management system, according to an embodiment;

FIG. 2 is a diagram for describing a structure of a processor of the predictive automated medical examination resource management system, according to an embodiment;

FIG. 3 is a flowchart of a method of predicting waiting days for a medical examination, according to an embodiment;

FIG. 4 is a flowchart of a method of predicting waiting days for a medical examination, according to another embodiment; and

FIGS. 5 to 8 are diagrams for describing a method of predicting waiting days for a medical examination, according to an embodiment.

FIG. 9 is a diagram illustrating an input data source of a predictive automated medical examination resource management system according to an embodiment of the disclosure;

FIGS. 10A and 10B are diagrams illustrating a core processing module of a predictive automated medical examination resource management system according to an embodiment of the disclosure;

FIG. 11 is a diagram illustrating an output and control target of a predictive automated medical examination resource management system according to an embodiment of the disclosure;

FIG. 12 is a diagram for describing a method of generating a predicted demand curve, according to an embodiment of the disclosure;

FIG. 13 is a diagram for describing a method of generating a predicted waiting day curve, according to an embodiment of the disclosure;

FIG. 14 is a diagram for describing a method of generating an automated control signal according to predicted waiting days, according to an embodiment of the disclosure;

FIGS. 15A, 15B and 15C are diagrams for describing an integrated operation method with an external system and hardware of the disclosure; and

FIG. 16 is a diagram for describing a cyclic operation method according to an embodiment of the disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As the disclosure allows for various changes and numerous embodiments, particular embodiments will be illustrated in the drawings and described in detail in the description. The effects and features of the disclosure, and ways to achieve them will become apparent by referring to embodiments that will be described later in detail with reference to the drawings. However, the disclosure is not limited to the following embodiments but may be embodied in various forms.

Hereinafter, embodiments will be described in detail with reference to the accompanying drawings, and in the description with reference to the drawings, like reference numerals denote like elements, and redundant description thereof will be omitted.

While such terms as “first” or “second,” etc., may be used to describe various elements, such elements must not be limited to the above terms. The above terms are used only to distinguish one element from another. Also, an expression used in the singular form encompasses the expression in the plural form, unless it has a clearly different meaning in the context. Also, it will be further understood that the terms “comprise” and/or “have” used herein specify the presence of stated features or elements, but do not exclude the presence or addition of one or more other features or elements.

In the drawings, for convenience of description, sizes of elements may be exaggerated or contracted. For example, since sizes and thicknesses of elements in the drawings are arbitrarily illustrated for convenience of explanation, the disclosure is not limited thereto.

In the embodiments below, it will be understood when a portion such as an area, an element, a unit, a block or a module is referred to as being “on” or “above” another portion, it can be directly on or above the other portion, or intervening portion may also be present. It will also be understood that when an area, an element, a unit, a block or a module is referred to as being “connected to” another area, element, unit, block or module, it can be directly connected to the another element, or it can be indirectly connected to the another element with another area, element, unit, block or module included therebetween.

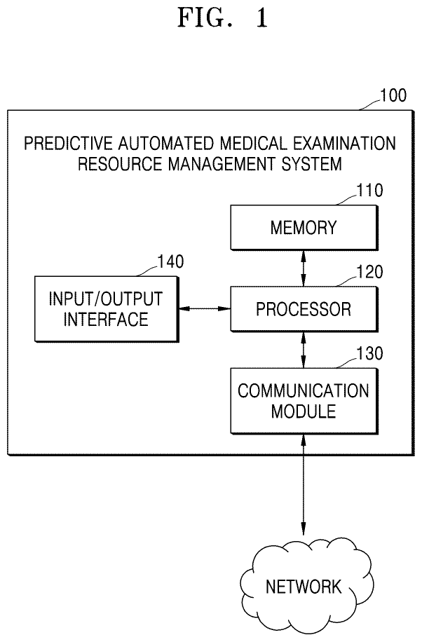

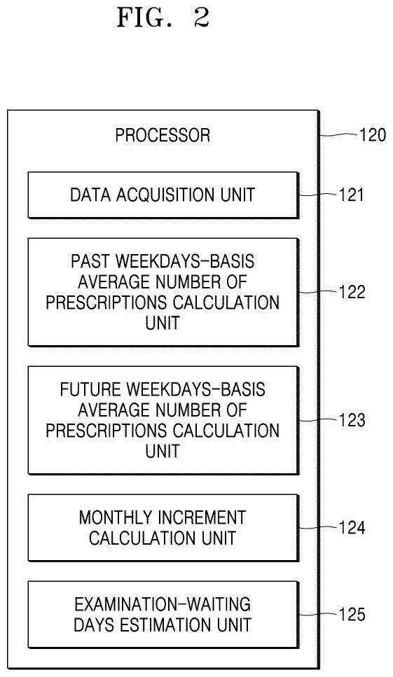

FIG. 1 is a diagram for describing the structure and operation of a predictive automated medical examination resource management system, according to an embodiment. FIG. 2 is a diagram for describing a structure of a processor of the predictive automated medical examination resource management system, according to an embodiment.

First, referring to FIG. 1, the predictive automated medical examination resource management system 100 according to an embodiment, may include a memory 110, a processor 120, a communication module 130, and an input/output interface 140. However, the disclosure is not limited thereto, and the predictive automated medical examination resource management system 100 may further include other components, or some components may be omitted. Some components of the predictive automated medical examination resource management system 100 may be divided into a plurality of devices, or a plurality of components may be merged into a single device.

The memory 110 may include a computer-readable recording medium, and may include a random access memory (RAM), a read-only memory (ROM), and a permanent mass storage device such as a disk drive. In addition, the memory 110 may temporarily or permanently store program code and a time-series prediction model for controlling the predictive automated medical examination resource management system 100. For example, the memory 110 may store examination prescription and execution data.

The processor 120 may acquire examination prescription and execution data. In addition, the processor 120 may calculate, based on the examination prescription and execution data, an average ratio of executed examinations for each day required from a prescription to the execution thereof and an average number of prescriptions based on past weekdays. In addition, the processor 120 may estimate, based on the average number of prescriptions based on past weekdays, an average number of prescriptions based on future weekdays for a predetermined period by using at least one time-series prediction model. Also, the processor 120 may calculate a monthly increment in the average number of prescriptions based on future weekdays compared to the average number of prescriptions based on past weekdays. Also, the processor 120 may allocate an expected number of prescriptions based on future weekdays based on the average number of prescriptions based on past weekdays and on the monthly increment. In addition, the processor 120 may estimate waiting days for an examination for each future date based on the expected number of prescriptions.

The communication module 130 may provide a function for communicating with an external server through a network. For example, a request generated by the processor 120 of the predictive automated medical examination resource management system 100, according to program code stored in a recording device such as the memory 110, may be transmitted to an external server through a network under the control of the communication module 130. Conversely, a control signal, a command, content, a file, etc. provided under the control of a processor of an external server may be received by the predictive automated medical examination resource management system 100, through the communication module 130 through a network. For example, a control signal or a command of an external server received through the communication module 130 may be transmitted to the processor 120 or the memory 110.

The communication method is not limited, and may include not only a communication method using a communication network that a network may include (e.g., a mobile communication network, wired Internet, wireless Internet, a broadcasting network) but also short-range wireless communication between devices. For example, the network may include any one of a personal area network (PAN), a local area network (LAN), a campus area network (CAN), a metropolitan area network (MAN), a wide area network (WAN), a broadband network (BBN), the Internet, etc. Further, the network may include, but is not limited to, any one or more of a network topology including a bus network, a star network, a ring network, a mesh network, a star-bus network, a tree or hierarchical network, and the like.

In addition, the communication module 130 may communicate with an external server through a network. Although the communication method is not limited, the network may be a short-range wireless network. For example, the network may include a Bluetooth, Bluetooth Low Energy (BLE), or Wi-Fi communication network.

In addition, the predictive automated medical examination resource management system 100 according to the disclosure may include the input/output interface 140. The input/output interface 140 may include a unit for an interface with an input/output device. For example, the input device may include a device such as a keyboard or a mouse, and the output device may include a device such as a display for displaying a communication session of an application. As another example, the input/output interface 140 may include a unit for an interface with a device in which functions for input and output are integrated into one, such as a touch screen. As a specific example, when processing a command of a computer program loaded in the memory 110, the processor 120 of the predictive automated medical examination resource management system 100 may display a service screen or content on a display through the input/output interface 140.

In addition, in other embodiments, the predictive automated medical examination resource management system 100 may include more components than those of FIG. 1. For example, the predictive automated medical examination resource management system 100 may be implemented to include at least some of the above-described input/output devices, or may further include other components such as a battery and a charging device for supplying power to internal components, various sensors, and a database.

Hereinafter, the internal configuration of the processor 120 of the predictive automated medical examination resource management system 100 according to an embodiment, will be reviewed in detail with reference to FIG. 2. For better understanding, it is assumed that the processor 120 to be described below is the processor 120 of the predictive automated medical examination resource management system 100 illustrated in FIG. 1.

The processor 120 of the predictive automated medical examination resource management system 100 may include a data acquisition unit 121, a past weekdays-basis average number of prescriptions calculation unit 122, a future weekdays-basis average number of prescriptions calculation unit 123, a monthly increment calculation unit 124, and an examination-waiting days estimation unit 125. According to some embodiments, components of the processor 120 may be selectively included or excluded from the processor 120. In addition, according to some embodiments, the components of the processor 120 may be separated or combined to express the functions of the processor 120.

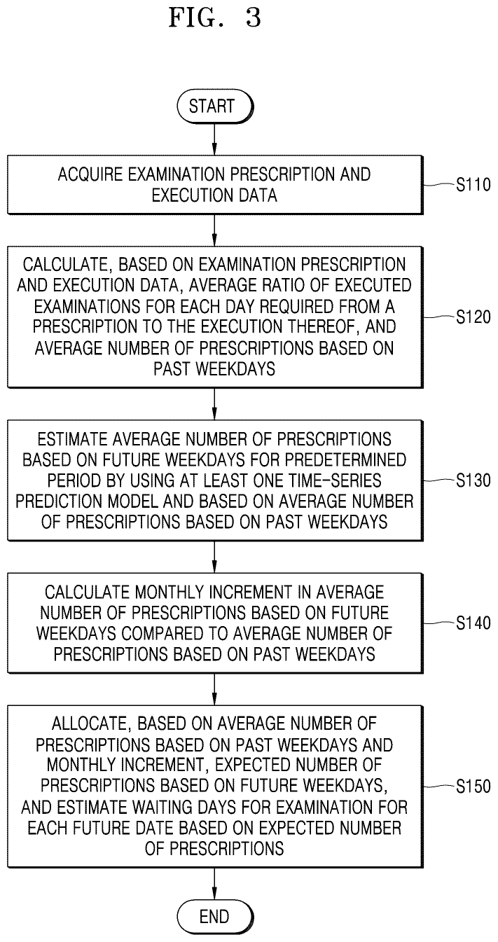

The processor 120 and the components of the processor 120 may control the predictive automated medical examination resource management system 100 so as to perform the operations included in the method of predicting waiting days for a medical examination, of FIG. 3 (S110 to S150). For example, the processor 120 and the components of the processor 120 may be implemented to execute instructions according to code of an operating system included in the memory 110 and code of at least one program. Here, the components of the processor 120 may be expressions of different functions of the processor 120, which are performed by the processor 120 according to a command provided by program code stored in the predictive automated medical examination resource management system 100. The internal configuration and certain operation of the processor 120 will be described with reference to a flowchart of the method of predicting waiting days for a medical examination, of FIG. 3.

FIG. 3 is a flowchart of a method of predicting waiting days for a medical examination, according to an embodiment.

Referring to FIG. 3, in operation S110, the processor 120 may acquire examination prescription and execution data.

The processor 120 according to an embodiment may distinguish a prescription department by reflecting a difference in examination prescription and execution patterns between weekdays and weekends. In addition, the processor 120 may acquire prescription data of prescriptions of a prescription department for a predetermined period, prescription data of prescriptions, for which examinations are conducted after the prescriptions, and execution data.

For example, the processor 120 may distinguish between prescription departments to reflect a difference in the patterns between weekdays and weekends. For example, the prescription departments may be divided into hemato-oncology and others. Also, the processor 120 may acquire execution data (e.g., including dates of execution and dates of prescriptions) for a certain past period (e.g., one year). Also, the processor 120 may acquire prescription data for a certain past period (e.g., one year).

In operation S120, the processor 120 may calculate, based on the examination prescription and execution data, an average ratio of executed examinations for each day required from a prescription to the execution thereof and an average number of prescriptions based on past weekdays.

The processor 120 according to an embodiment may calculate the number of prescriptions by date for each prescription department based on the execution data. Also, the processor 120 may calculate, based on the prescription data and the number of prescriptions by date, an average ratio of executed examinations for each day required from a prescription to the execution thereof. Also, the processor 120 may calculate an average number of prescriptions based on past weekdays, by dividing the number of prescriptions for a predetermined period by the number of weekdays.

For example, the processor 120 may calculate the number of cases per day, as to after how many days an examination is made after the date of prescription (e.g., 3 days) for each of prescription departments distinguished to reflect the pattern difference between weekdays and weekends (e.g., hemato-oncology and other departments). Subsequently, the processor 120 may calculate an average ratio of executed examinations for each day required from a prescription to the execution thereof, by dividing each number of cases by the total number of cases. For example, in order to increase the accuracy of a prediction model for waiting days for an examination for each future date, the processor 120 may limit data used when calculating the average ratio of executed examinations for each day required from a prescription to the execution thereof, to past data that is similar to the waiting days at the present time (e.g., 30 days). Also, the processor 120 may calculate the average number of prescriptions based on weekdays by dividing the number of prescriptions for a certain past period (e.g., 10 months) by the number of weekdays during the corresponding period.

In operation S130, the processor 120 may estimate an average number of prescriptions based on future weekdays for a predetermined period by using at least one time-series prediction model and based on the average number of prescriptions based on past weekdays.

The processor 120 according to an embodiment may estimate the average number of prescriptions based on future weekdays by using an ensemble model in which result values of a plurality of time-series prediction models are combined.

For example, the processor 120 may obtain an average number of prescriptions based on weekdays for each month for a certain past period (e.g., 7 years) by prescription departments divided to reflect the pattern difference between weekdays and weekends (e.g., hemato-oncology and other departments). Subsequently, the processor 120 may calculate an average number of prescriptions based on monthly weekdays for a certain future period (e.g., 9 months) through at least one time-series model. For example, the processor 120 may estimate the average number of prescriptions based on future weekdays by using an ensemble model that combines and utilizes a plurality of time-series models, in order to increase the accuracy of predicting the average number of prescriptions based on future weekdays.

In operation S140, the processor 120 may calculate a monthly increment in the average number of prescriptions based on future weekdays compared to the average number of prescriptions based on past weekdays.

For example, the processor 120 may derive an increment in the average number of prescriptions based on weekdays for each month for a certain future period (e.g., 9 months) (e.g., an expected increment in the number of prescriptions after 1 month compared to the past 10 months: 1.016794 (Hemato-oncology), 1.014449 (Others), an expected increment in the number of prescriptions after 2 months compared to the past 10 months: 1.016794 (Hemato-oncology), 1.005481 (Others), an expected increment in the number of prescriptions after 3 months compared to the past 10 months: 1.102290 (Hemato-oncology), 1.039362 (Others), etc.) compared to the average number of prescriptions based on weekdays for a certain period in the past (e.g., 10 months) to be used in calculation of waiting days.

In operation S150, the processor 120 may allocate, based on the average number of prescriptions based on the past weekdays and the monthly increment, an expected number of prescriptions based on future weekdays, and estimate waiting days for an examination for each future date based on the expected number of prescriptions.

The processor 120 according to an embodiment may set additional capacity that can be operated in addition to the capacity if the date of examination requiring a reservation is a weekday. In addition, when the estimated number of reservations for the date of examination exceeds a sum of the capacity and the additional capacity, the processor 120 may distribute the excess capacity to a date prior to the date of examination according to predetermined conditions.

The processor 120 according to an embodiment may calculate, as waiting days for each future date, a section until a first day when two consecutive weekdays start to appear, on which the number of reservations compared to the capacity for each future date is less than or equal to a predetermined ratio. For example, the processor may obtain reservation data at the present time and capacity data expected to be operated for a certain period in the future (e.g., 9 months) by date. In addition, the processor 120 may allocate the expected number of prescriptions for the corresponding month based on future weekdays. For example, the processor 120 may multiply the average number of prescriptions for a certain period in the past by an increment in the average number of prescriptions by month for a certain period in the future.

In addition, the processor 120 may update the waiting days for each future date by adding, to a reservation data value of each future date, a value obtained by multiplying the average number of prescriptions for a certain period in the past by a rate at which execution is to be made for each future date when a prescription is issued on the weekday. For example, the processor 120 may set the additional capacity that can be additionally operated on weekdays in addition to the capacity. In addition, when there are more reservations than the capacity on non-weekdays or more reservations than the sum of the capacity and the additional capacity on weekdays, the processor 120 may sequentially fill the excess reservations from earlier dates where the reservations are not yet full. In this case, the processor 120 may repeat the distribution operation until the excess reservations are eliminated, by distinguishing between a prescription department (e.g., hemato-oncology department) for which the excess reservations are distributed on any days where reservation is not full, regardless of whether it is a weekday, and a prescription department (e.g., other departments) for which the excess reservations are distributed only on weekdays where reservation is not full.

In addition, the processor 120 may calculate and output waiting days for each future date. For example, the processor 120 may calculate, as waiting days, a section until a first day when two consecutive weekdays start to appear, on which the number of reservations compared to the capacity is equal to or less than a certain standard (e.g., 95%) for each inquired future date. In addition, the processor 120 may visualize and express the expected number of waiting days by date until a certain future date (e.g., 9 months later) derived from the current time, as a graph or the like.

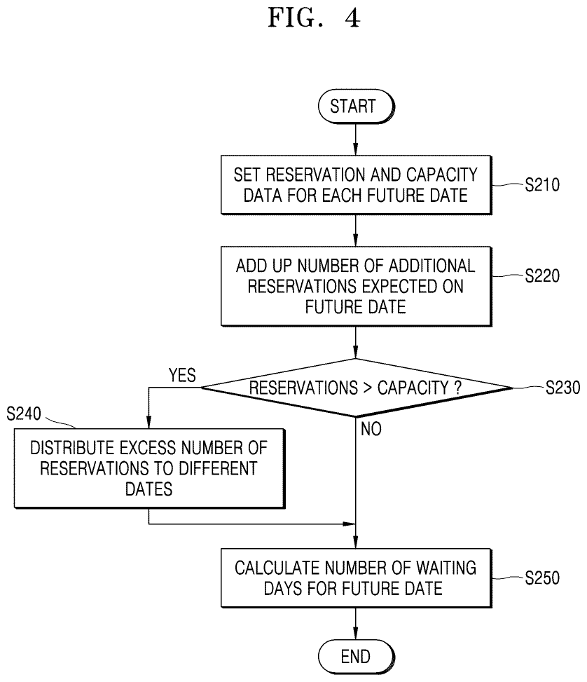

FIG. 4 is a flowchart of a method of predicting waiting days for a medical examination, according to another embodiment.

Referring to FIG. 4, in operation S210, the processor 120 may set reservation and capacity data for each future date. For example, the processor 120 may secure reservation and capacity data corresponding to a future date from the present time by distinguishing between weekdays or non-weekdays.

In operation S220, the processor 120 may add up the number of additional reservations expected on a future date. For example, the processor 120 may divide data into prescription departments so as to distinguish between weekday and weekend patterns, and calculate the number of executions and prescriptions by date. For example, the processor 120 may predict the number of prescriptions by month in the future by using a time-series pattern of the number of prescriptions. In addition, the processor 120 may calculate the number of additional reservations expected for a future date by multiplying the number of prescriptions predicted for each month by the execution rate by date. In addition, from among the number of excess reservations exceeded when the expected number of additional reservations is added to the number of existing reservations on future dates, the processor 120 may first add the expected number of additional reservations for a prescription department, to which the reservations may be distributed regardless of whether the days is a weekday or not, to the number of existing reservations, and then add the expected number of additional reservations for a prescription department, to which the reservations may be distributed only for weekdays.

In operations S230 and S240, the processor 120 may distribute the excess number of reservations to different dates. For example, for weekdays, the processor 120 may determine that the number of reservations is exceeded when the number of reservations is greater than the sum of the capacity and additional persons. For example, in the case of a prescription department for which the excess number of reservations can be distributed regardless of whether or not it is a weekday, the processor 120 may sequentially distribute the number of reservations that exceed due to the addition of the expected number of reservations to the number of existing reservations, from the earlier dates on which the number of reservations is not exceeded, regardless of whether it is a weekday or not. Also, in the case of a prescription department for which the excess number of reservations can be distributed only on weekdays, the processor 120 may sequentially distribute the number of reservations that exceed due to the addition of the existing number of reservations to the number of existing reservations, from the earlier dates on which the number of reservations is not exceeded only on weekdays. For example, the processor 120 may repeat the process of distributing the excess number of reservations until there is no more excess number of reservations.

In operation S250, the processor 120 may calculate the number of waiting days for the future date. For example, the processor 120 may calculate and display, as waiting days, a period until a first day on which two consecutive weekdays are repeated, on which the number of reservations for each inquired future date does not exceed a certain standard (e.g., 95%) compared to the capacity.

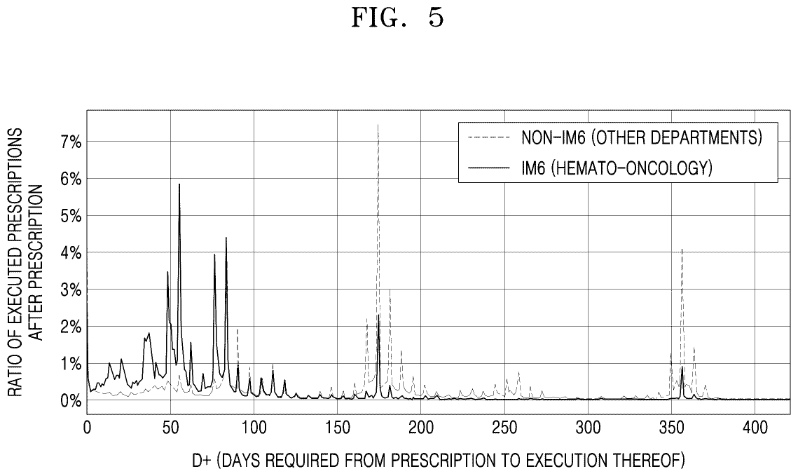

FIGS. 5 to 8 are diagrams for describing a method of predicting waiting days for a medical examination, according to an embodiment.

First, referring to FIG. 5, the number of prescriptions for a daily average CT scan examination based on weekdays from January to October 2021 is 541 cases for non-IM6 and 250 cases for IM6. Here, non-IM6 denotes other prescription departments except hemato-oncology, and IM6 denotes hemato-oncology. In addition, an increase rate of monthly prescriptions from November 2021 to July 2022 compared to January to October 2021 may be calculated.

Referring to FIG. 5, the distribution of the number of reservations to be additionally made from D+0 days to D+1448 days with respect to the day on which a prescription is issued may be calculated as follows.

Non-IM6: 541 cases×prescription increase rate for the corresponding month between November 2021 and July 2022 (e.g., November 2021:1.014449)×distribution ratio of the number of executions from D+0 to D+1448 during January to October 2021.

IM6: 250 cases×prescription increase rate for the corresponding month between November 2021 and July 2022 (e.g., November 2021:1.016794)×distribution ratio of the number of executions from D+0 to D+1448 during January to October 2021.

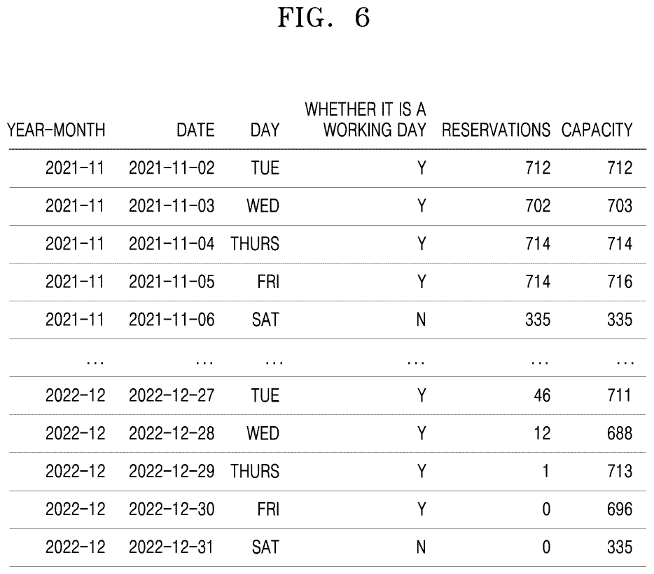

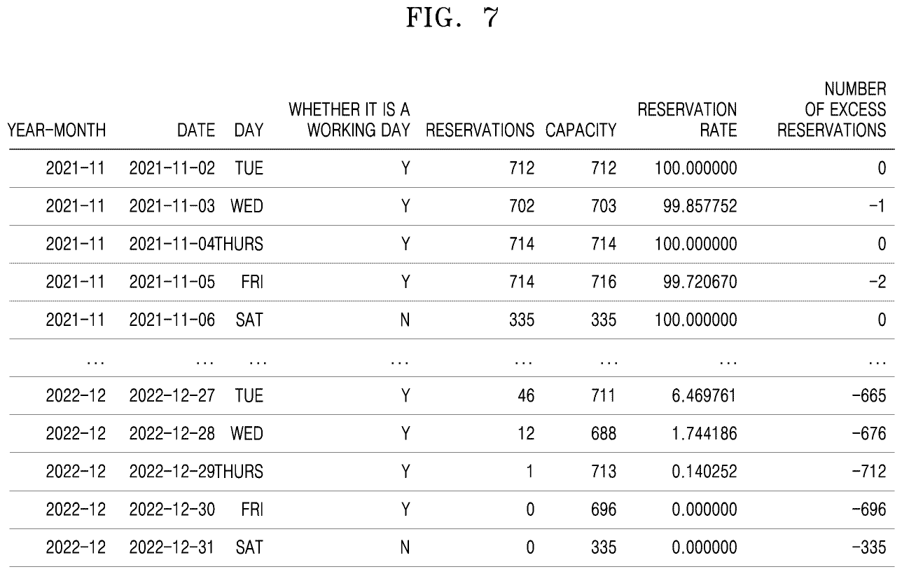

FIGS. 6 and 7 are diagrams for describing a method of calculating reservation data for calculating waiting days for each future date by reflecting the additional capacity, according to an embodiment.

First, referring to FIG. 6, a processor may add, to the inquired reservations on the day (e.g., Nov. 2, 2021) compared to the capacity, the number of reservations in consideration of prescriptions to be issued on weekdays from D+1 (e.g., Nov. 3, 2021). For example, the processor may set the available additional capacity to 10 in addition to the capacity. In addition, the processor may set the capacity in consideration of various situations. Also, the processor 120 may calculate a reservation rate (reservation/capacity) and excess reservations (reservation-capacity) by reflecting the set capacity. For example, the processor 120 may modify the additionally combined reservation information by reflecting the additional capacity setting. For example, referring to FIG. 7, when the item indicating whether the day is a working day is N (not applicable (N)), and the excess reservations are greater than 0, the processor 120 may sequentially move the excess reservations in order from a date where the excess reservations are less than 0. In addition, when the item indicating whether the day is a working day is Y (applicable (Y)), and the number of excess reservations is greater than the available additional capacity, the processor 120 may sequentially move the excess reservations exceeding the available additional capacity, in order from a date where the excess reservations are less than 0. For example, the processor 120 may fill reservations regardless of weekdays in the case of hemato-oncology, and fill reservations only if it is a weekday in case of non-hemato-oncology (other departments).

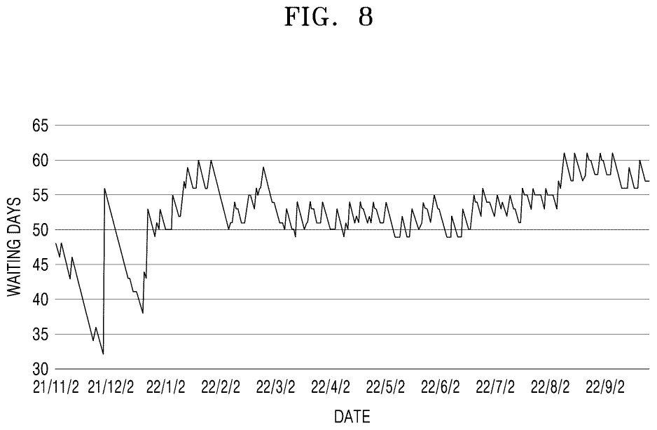

FIG. 8 is a diagram showing a result of prediction of waiting days for an examination, according to an embodiment. For example, FIG. 8 shows a result of prediction of waiting days for an examination, according to FIG. 7. In detail, FIG. 8 shows a result of estimating waiting days for an examination for each future date compared to the present time (Nov. 2, 2021).

The disclosure may be utilized when calculating waiting days for various examinations requiring a reservation at a hospital. When waiting days for various examinations may be accurately calculated from a future date, as according to the disclosure, factors that affect a delay in treatment may be minimized by securing appropriate investment and operation plans for examination equipment in advance. In addition, the disclosure may be applied effectively in hospitals where the demand from patients for examinations is greater than the available capacity of examinations. In addition, the disclosure may be applied to examinations, for which a prescription is issued on weekdays but which are conducted also on days that are other than weekdays.

In addition, according to the disclosure, waiting days from a future time may be provided compared to the method according to the related art, where only waiting days at the present time are provided. In addition, the pattern of the number of reservations to be filled in the future may be elaborately and accurately predicted.

In addition, according to the disclosure, the accuracy of predicting the number of waiting days for a future date may be improved. In addition, in order that a difference in practice patterns between weekdays and weekends can be distinguished, patient groups may be classified according to prescription departments and applied differently. In addition, a past execution pattern having a pattern similar to the aspect of the waiting days at the current time may be set to be utilized. In addition, a time-series ensemble model may be used to accurately predict the number of future prescriptions. In addition, in the case of weekdays, it is possible to increase the level of reality by reflecting the additional capacity that can be additionally operated, and the excess reservations exceed the capacity may be efficiently distributed according to prescription departments and whether it is a weekday or not.

In addition, according to the disclosure, as it becomes possible to predict waiting days not only for the present time but also for future dates, various operational attempts such as changing work scheduling may be made in the short term in order for hospitals to effectively manage waiting days, and in the long term, the predicted waiting days may be used in decision-making when deciding preemptive equipment investment. In addition, for patients, it is possible to schedule an examination in a timely manner.

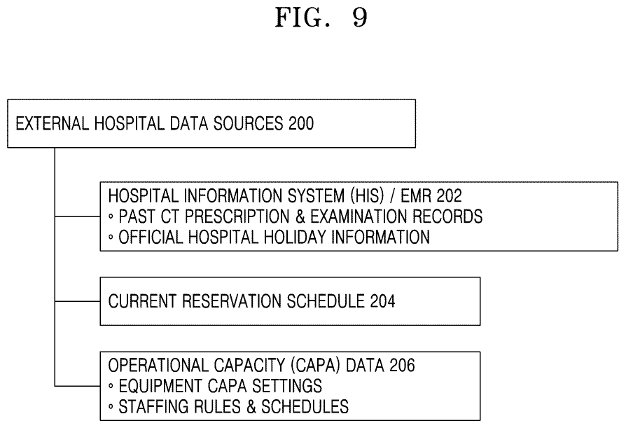

Referring to FIGS. 9 to 11 together, a predictive automated medical examination resource management system 100 according to an embodiment of the disclosure includes an automated resource management module 300. For example, the automated resource management module 300 may include a predictive prescription forecasting engine 310, a dynamic waiting day simulation system 320, and an automated resource control framework 340.

The predictive prescription forecasting engine 310 according to the disclosure predicts future CT examination demand with very high accuracy through an advanced machine learning pipeline. This serves to generate the most basic and important input data for all subsequent actions of the system.

The dynamic waiting day simulation system 320 according to the disclosure precisely simulates expected waiting days for each day at a specific point in the future by combining predicted demand data with the actual operating conditions of a hospital. This is a key process that transforms abstract demand forecasts into concrete, actionable operational metrics.

The automated resource control framework 340 according to the disclosure autonomously executes actions for hospital operation and strategic resource allocation according to predefined rules based on simulation results. This is the final step in the system, which connects predicted insights to actual operational improvements.

The integration of these three components, the predictive prescription forecasting engine 310, the dynamic waiting day simulation system 320, and the automated resource control framework 340, overcomes the limitations of existing decision support systems. While existing systems merely provide analysis information to human managers to assist their judgment, the present system has the characteristics of a cyber-physical control system that directly controls other computer systems (e.g., scheduling systems, enterprise resource planning (ERP), human resources management (HRM)) and physical hardware (CT scanners) based on analysis results. This opens up new possibilities for technology to move beyond simply providing information and intelligently automate hospital operational processes themselves, minimizing human intervention and enabling data-driven, optimal actions to be taken automatically, in real time, and over the long term.

The architecture of the present system is designed to follow a clear flow of data acquisition, processing, and automated action execution. The core components of the system include the processor 120, the memory 110, and the communication module 130, which enable smooth data exchange and control with external hospital systems.

Referring to FIG. 9, as an input, the system according to the disclosure begins by collecting raw data from the core database of the hospital, i.e., electronic medical records (EMR) or hospital information system (HIS). This data includes past CT prescriptions and actual examination records accumulated over several years, official holiday information of the hospital, and the current confirmed appointment schedule.

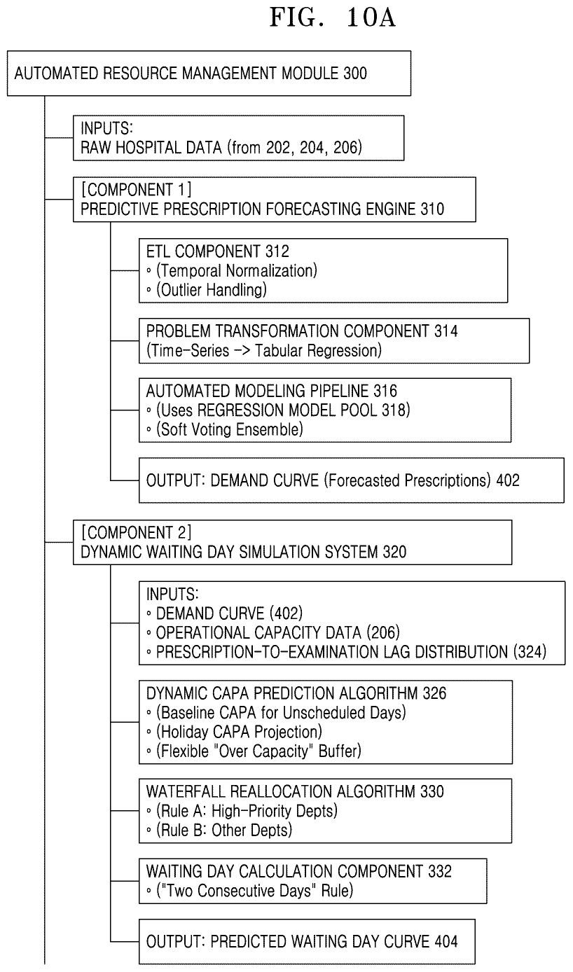

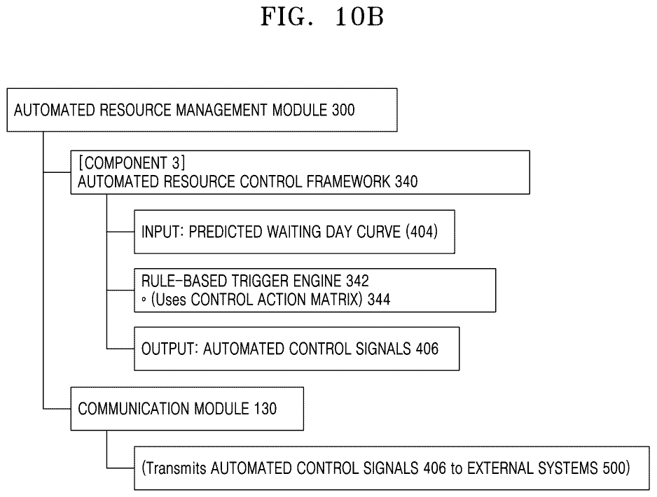

Referring to FIGS. 10A and 10B, in processing, the collected raw data is first transferred to the predictive prescription forecasting engine 310. The above engine refines data and uses machine learning models to generate demand curves that represent future trends in the average number of daily prescriptions. This demand curve is used as a key input value for the next step, the dynamic waiting day simulation system 320. The simulation system combines this demand curve with CT scan capacity (CAPA) data of the hospital to produce a predicted waiting day curve which is a calculation of the expected waiting days for each future date.

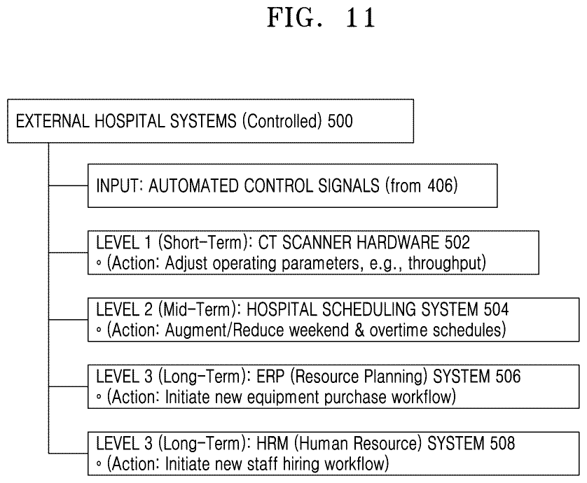

Referring to FIG. 11, in Output & Action, a final generated predicted waiting day curve is continuously monitored by the automated resource control framework 340. This framework transmits an automated control signal to an external system via the communication module 130 when the predicted waiting days exceed or fall below a predetermined threshold. This control action goes beyond simply displaying information on a screen, but directly drives other core systems in the hospital. In the short term, the operating parameters of the CT scanner hardware is adjusted, in the medium term, the work schedule of the hospital scheduling system is modified, and in the long term, a new equipment purchase workflow is automatically initiated in the hospital's enterprise resource planning (ERP) system and a new staff hiring workflow is automatically initiated in the human resources management (HRM) system.

These data flows form a complete closed-loop: sensing (data collection), prediction (demand forecasting and waiting days simulation), and execution (automated control). This structure clearly demonstrates that the disclosure is not a simple prediction technology, but a concrete and tangible technical implementation that fundamentally improves the operational efficiency of a complex system such as hospitals. This demonstrates that the disclosure is, beyond abstract ideas or business methods for organizing human activity, a clear technological disclosure that combines computer hardware and software to solve specific problems.

The prediction accuracy of the present system is the most important foundation that determines the reliability of the entire automation framework. Thus, the predictive prescription forecasting engine 310 is designed as a sophisticated and automated machine learning pipeline that goes beyond simply using a single model and maximizes the characteristics of the data and minimizes model uncertainty.

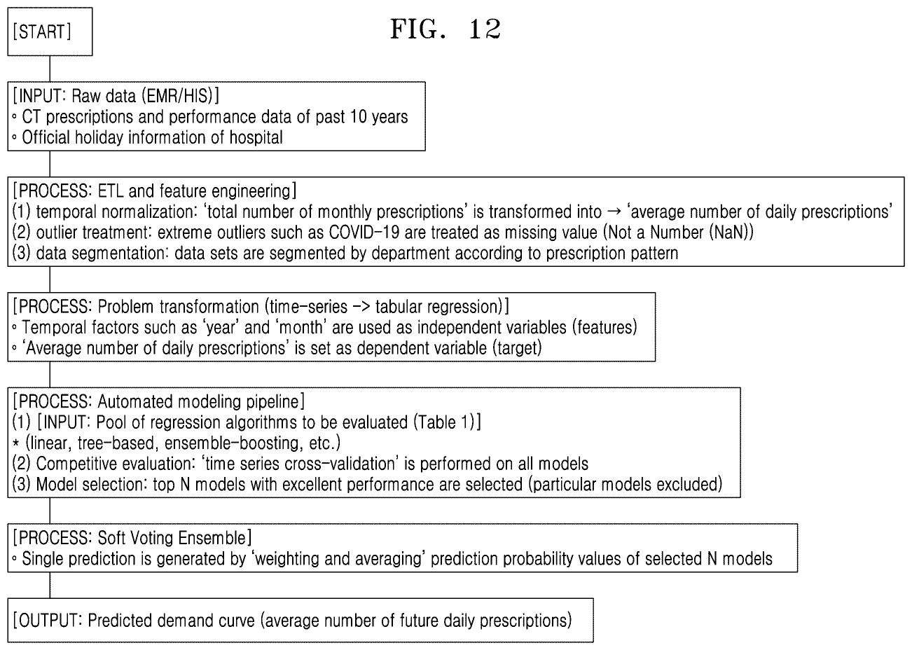

FIG. 12 is a diagram for describing a method of generating a predicted demand curve, according to an embodiment of the disclosure.

The first step to achieving high-quality prediction results is a systematic data collection, transformation, and feature engineering Extract, Transform, Load (ETL) process that processes raw data into a form suitable for modeling.

For example, referring to FIG. 12, a predictive model extracts a large amount of historical CT prescription and performance data spanning approximately 10 years from a hospital database to learn long-term trends and seasonality. Leveraging such long-term data is essential for the model to understand macroscopic patterns beyond short-term fluctuations.

For example, one of the core feature engineering techniques of the above engine is to convert raw, total number of monthly prescriptions into a normalized metric called ‘average number of daily prescriptions’ by dividing the same by the actual number of business days in the month (excluding weekends and holidays), rather than using the average number as is. This process of removing data distortion caused by different business days in each month plays a crucial role in enabling the model to learn pure time-series patterns more clearly.

The quality of data directly affects the performance of the model. Extreme outliers, such as those caused by the COVID-19 pandemic in March 2020, can introduce significant bias into model training. Instead of simply removing data from these specific periods, the present system treats the data as missing values (Not a Number (NaN)) and induces the model to estimate (imputate) the values in that interval by using statistical techniques. This is a sophisticated data refinement strategy that minimizes the negative impact of outliers while maintaining data continuity.

Considering that not all CT prescriptions follow the same pattern, the present system segments the data into specific departments (e.g., hematology and oncology) that exhibit distinct differences in weekday and weekend prescription and testing patterns, and other departments. Dividing the patient population into homogeneous subgroups when training each model is an important strategy that significantly improves the granularity and accuracy of predictions.

The most unique technical feature of the forecasting engine lies in the novel approach that breaks away from traditional time series analysis methodologies (e.g., ARIMA, Prophet).

According to the system of the disclosure, a time-series problem of predicting changes in the number of prescriptions over time is transformed into a tabular regression problem of predicting a dependent variable using independent variables. To this end, by using temporal elements such as ‘year’ and ‘month’ as categorical features, and the ‘sequence’ index indicating the order of time as a continuous feature, influence on the dependent variable, the target variable, ‘average number of daily prescriptions’ is modeled.

This approach has been experimentally identified to outperform traditional time-series models in the domain of interest, namely the problem of predicting the average number of daily prescriptions per month in a hospital, as measured by key performance metrics such as root mean square error (RMSE) and mean absolute percentage error (MAPE). This suggests that the complex seasonality, trends, and influence of external factors inherent in time series data may be more effectively captured within a tabular data structure. Recent trends in machine learning research have also shown that foundation models for general tabular data are being applied to time series prediction to achieve superior performance, supporting the technological advancement and validity of the approach of the disclosure.

According to the disclosure, prediction is performed by dividing the entire data into ‘departmental segmentation’. In certain departments or on weekends/holidays, the average number of daily prescriptions frequently approaches ‘0’ or is a very small integer. In this case, there is limitation in that the actual performance of the model cannot be properly evaluated because the error rate diverges infinitely or becomes excessively distorted as the denominator of the MAPE approaches 0. Additionally, hospital data may contain ‘outliers’ caused by abnormal external factors such as the COVID-19 pandemic or emergency situations. RMSE is calculated by squaring the error, and thus penalizes these temporary spike values too heavily. This leads the model to overfit to rare extreme values rather than general trends, which reduces overall predictive stability.

Rather than relying on a single, specific algorithm, this engine employs an automated pipeline that dynamically finds and combines the optimal models for a given data set, maximizing prediction accuracy and reliability.

The system of the disclosure performs automated learning and performance evaluation on all models within a predefined, extensive pool of regression algorithms. This process is based on the time-series cross-validation technique that considers the characteristics of time-series data, and objectively evaluates the performance of each model in future predictions.

According to the disclosure, when evaluating a model within a regression algorithm pool in a competitive algorithm evaluation stage, a mean absolute error (MAE) may be set as a primary sorting metric instead of the existing RMSE or MAPE. According to MAE-based upper model extraction according to the disclosure, MAE values of respective models, calculated through time series cross-validation, are compared, and the top N models with the smallest absolute amount of error are selected as ensemble candidates.

$$MAE = \frac{1}{n} {\sum }_{i = 1}^{n} \vert {y}_{i} - {\hat{y}}_{i} \vert \$ MAE$$

(where yᵢ is the actual number of prescriptions, ŷᵢ is the number of predictions).

Once the evaluation of all models is complete, the system selects a preset number of the top N models (e.g., 10) with the best performance. However, certain models known to destabilize the overall forecast trend (e.g., lar-Least-Angle Regression) are excluded from this process. Finally, rather than simply deciding the prediction results of the selected models by majority vote, the results are combined through a ‘soft voting’ method that weights and averages the predicted probability values of each model. These ensemble techniques have the effect of compensating for the biases or errors that a single model may have, thereby producing a much more stable and accurate final prediction, and this is a widely proven methodology in the field of machine learning.

The prediction results of the present system are processed by mapping the same 1:1 with physical CT examination slots (CAPA) in the subsequent stage, the ‘dynamic waiting day simulation system’. For example, unlike MAPE, which represents a ratio (%), or RMSE, which has a square root value, MAE may intuitively represent a physical quantity such as “how many slot differences occur on average per day.” This provides a direct and clear basis for determining the buffer size (e.g., 3 cases) based on the predicted MAE value (e.g., 2.5 cases) when the simulation system sets an ‘Over Capacity Buffer’. Additionally, even in segmented datasets by department, where the actual number of prescriptions is relatively small, MAE provides consistent performance indicators without distortion. This enables the construction of a stable ensemble model even for rare medical departments with small amounts of data. Additionally, an automated resource control framework may directly control hardware and personnel when predicted waiting days exceed a threshold. Models selected based on MAE are less susceptible to outliers and more accurately reflect the overall operating trend, minimizing the risk of false positives, where temporary data spikes cause the system to send unnecessary control signals (e.g., incorrect equipment parameter changes).

According to the disclosure, considering the domain specificity of CT examination resource management (physical slot constraints, small-scale clinical department data, presence of intermittent outliers), MAE is adopted as an optimal reference point for model selection. This goes beyond simply changing statistical indicators, but is an essential technical feature for ensuring the accuracy of simulations and the stability of automated control systems.

Existing time series forecasting models (e.g., ARIMA) have a strong tendency to overfit when forecasting the distant future (e.g., one year later) in a medical data environment with high data scarcity and irregular outliers such as pandemics, and thus have technical limitations in that forecasting accuracy is reduced. Thus, to technically resolve this overfitting problem and make the error rate of long-term time series forecasting within an acceptable range, time series data was converted into a tabular regression problem and an ensemble model based on MAE was applied.

The entire process of ETL, tabular regression transformation, competitive evaluation, and ensemble model building described above is repeated individually for each of the multiple key prediction target indicators, including total prescriptions and valid prescriptions, for the entire hospital and each major prescribing department (e.g., hematology-oncology, surgery, gastroenterology, urology, thoracic surgery, and pulmonology). This indicates that the system is able to automatically generate customized predictive models optimized for data streams with different characteristics, ensuring the scalability and accuracy of the overall system.

The design of these predictive engines is a necessary choice to satisfy the extremely high level of reliability required by subsequent automated control actions. Incorrect predictions can have serious consequences, such as unnecessary purchases of millions of dollars of equipment or inefficient use of personnel. Thus, the present system possesses technological originality in that, beyond finding the best single model, an ‘automated machine learning pipeline’ itself that minimizes prediction uncertainty by leveraging the collective intelligence of various models is realized. The high reliability of this prediction engine is a prerequisite for ensuring the feasibility of the closed-loop control framework, which is the core of the disclosure.

TABLE 1
Pool of regression algorithms to be evaluated for
automated model selection in prediction engines

| Model classification | Regression algorithm to be evaluated |
|---|---|
| Linear Models | Linear Regression, Lasso Regression, Ridge |
| | Regression, ElasticNet, Least Angle |
| | Regression (LARS), Bayesian Ridge, |
| | Stochastic Gradient Descent (SGD), Robust |
| | Regression (RANSAC, Theil-Sen, Huber) |
| Tree-based model | Decision Tree |
| Ensemble - Bagging | Random Forest, Extra Trees |
| Ensemble - Boosting | AdaBoost, Gradient Boosting, LightGBM, |
| | XGBoost, CatBoost |
| Other models | K-nearest neighbors (KNN), support vector |
| | machines (SVM), and multi-layer |
| | perceptron (MLP) |

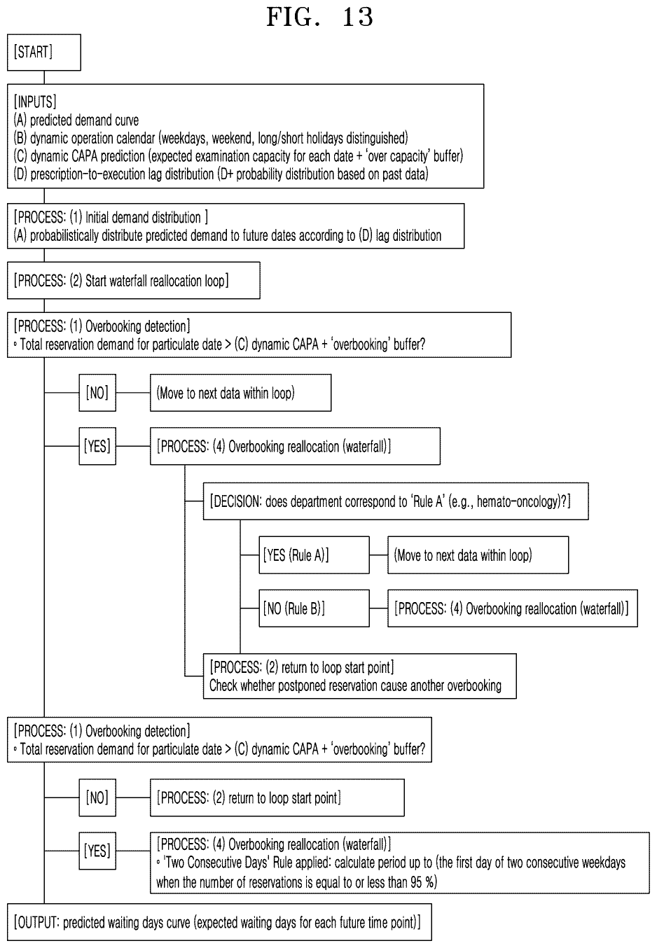

FIG. 13 is a diagram for describing a method of generating a predicted waiting day curve, according to an embodiment of the disclosure.

When the predictive prescription forecasting engine 310 predicts ‘how much’ demand will occur, the dynamic waiting day simulation system 320 simulates ‘how’ that demand will be handled under the hospital's operating constraints, resulting in ‘how many days’ of waiting days. Instead of a simple mathematical queue model, the predictive prescription forecasting engine 310 and the dynamic waiting day simulation system 320 function as a customized digital twin that precisely reflects the complex and nonlinear rules of hospital operations.

For accurate simulation, it is necessary to faithfully implement real-world constraints in the data model.

For example, referring to FIG. 13, the system of the disclosure generates a master calendar that specifies whether each day is a weekday, a weekend, a short holiday (1-2 days), or a long holiday (3 days or more, such as Lunar New Year or Chuseok) for dates in the next two years. In particular, distinguishing between short and long holidays is an important technical detail. During long holidays, the available examination capacity (CAPA) is significantly reduced due to difficulties in adjusting the workforce, whereas during short holidays, the reduction in capacity is relatively small, and thus modeling these separately greatly increases the realism of the simulation.

The system of the disclosure analyzes past data to calculate a probability distribution of how many days it took from the date a CT prescription was issued until the actual examination was performed (D+ days). For example, statistical patterns such as ‘proportion of tests performed within 30 days of prescription’ and ‘proportion of tests performed within 90 days’ are used as key inputs to simulate the temporal distribution of new prescriptions expected to occur in the future and how the prescriptions will fill appointment slots.

Another key input to the simulation is the available examination capacity, or CAPA, for each future date. The system includes a dynamic algorithm to reasonably estimate future CAPA, which has not yet been scheduled.

For future weekdays and weekends, the system may set a default CAPA through two options. First, an administrator in charge of each CT equipment may apply values predefined in advance, or second, a user may choose to use the past average or moving average values by day of the week. This flexibility allows the system to adapt to different operating environments.

For future holidays, the CAPA patterns of similar holidays that occurred in the past year (e.g., Children's Day last year) are analyzed and the median value thereof is applied. Especially for long holidays, sophisticated logic to set CAPA differently by reflecting the characteristics of each day within the holiday (e.g., the first and last days of a three-day holiday) is included.

The system of the disclosure defines a separate buffer called ‘Over Capacity’, which is an additional number of persons (e.g., 30 cases) that may be accommodated for emergency/critical patients or new patients, even if the official capacity (CAPA) is full on weekdays. This concept is a key element in simulating realistic flexibility in hospital operations, preventing overly long waiting days and improving the accuracy of predictions.

A waterfall reallocation algorithm according to the disclosure is the heart of the simulation engine and is the core logic that simulates how reservations are reallocated when the predicted demand exceeds capacity.

In a first stage, the Initial Demand Allocation stage, it is assumed that on each day (e.g. today) within a simulation period, as many ‘virtual prescriptions’ as predicted by the predictive prescription forecasting engine are issued. These virtual prescriptions are probabilistically distributed to several future dates based on ‘prescription-to-execution lag distribution’ data to form an initial reservation demand.

In a second stage, the overbooking detection (overflow detection) stage, the system of the disclosure compares the total reservation demand distributed for each date with the predicted CAPA for that date (including the ‘over capacity’ buffer for weekdays) to identify the days on which the capacity is exceeded and the number of persons in excess of that date.

In a third stage, when overbooking is detected in the Department-Specific Overflow Handling (The “Waterfall”) stage, the system of the disclosure applies a “Waterfall” method in which the corresponding number of persons are sequentially pushed to a next available reservation date. This process demonstrates sophistication in that not all patients are treated equally, but rather two differentiated rules are applied depending on the characteristics of the department.
Rule A (specific departments such as hematology-oncology): Reflecting the characteristics of high-urgency patient groups, such as those requiring anticancer treatment, overbooked appointments are reallocated to a next available appointment date, regardless of whether it falls on a weekday or weekend. This builds clinical flexibility into the system logic.
Rule B (all other departments): To reflect the characteristics of routine follow-up examinations, etc., overbooked appointments are rescheduled to a next available appointment date on a weekday.

When the overbooking is reallocated to another date in an iteration stage as a fourth stage, another overbooking may occur on that date. Therefore, this detection and reallocation process is repeated throughout the simulation period until no more overbooking occurs.

After all reservation reallocations are completed, the system of the disclosure calculates a final waiting day index. For example, the “Two Consecutive Days” Rule applies. The waiting days for a certain date is not simply a period until the first day a reservation becomes available. The system calculates, as waiting days, a period up to ‘the first day of two consecutive weekdays when the number of reservations compared to the number of seats available for each future date being searched is equal to or less than a certain standard (e.g. 95%)’. This rule is an important device to prevent statistical errors in which waiting days are calculated too short due to temporary vacancy on only one day, and to evaluate the load status of the system more reliably and realistically.

The value of this simulation system lies in the realism of predictions. By embedding the complex rules of hospital operations-such as different scheduling policies for different departments, variable operating capacities based on holiday types, and flexible buffers for unexpected patients-into the algorithm, the system may predict potential future bottlenecks and their ripple effects with very high fidelity. These highly reliable simulation results go beyond simple average wait time predictions and serve as a bridge, providing a strong basis for automating critical business decisions, such as billions of won in capital investments or large-scale hiring.

The most fundamental originality of the disclosure lies in the fact that insights gained through prediction and simulation are not simply provided as information, but rather a closed loop that directly and autonomously controls the operation of the hospital system is completed based on this information. This automated control framework uses predicted waiting days as a key input signal to control the system over a range of time horizons, from short-term operational optimization to long-term capital investment.

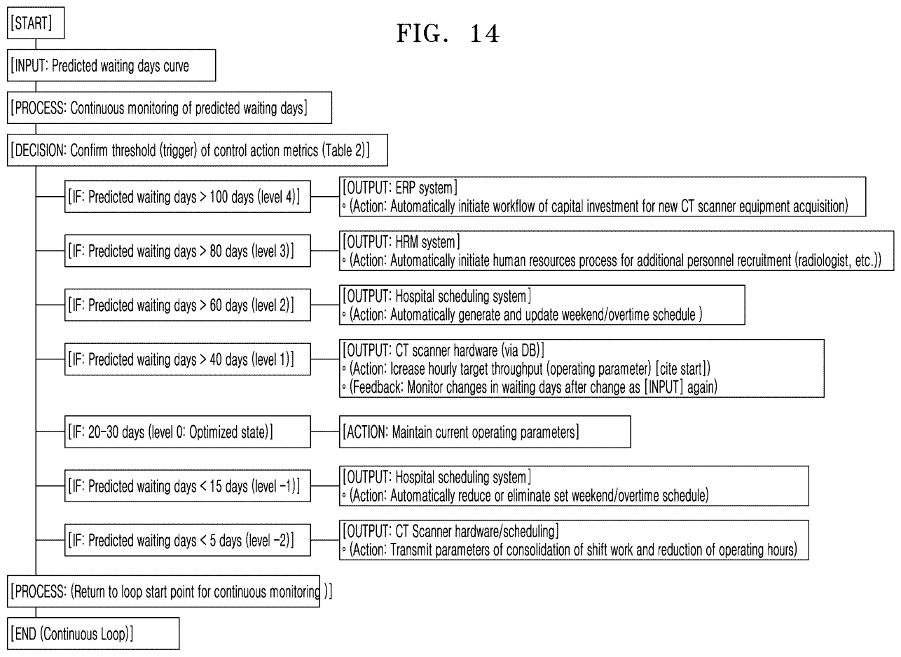

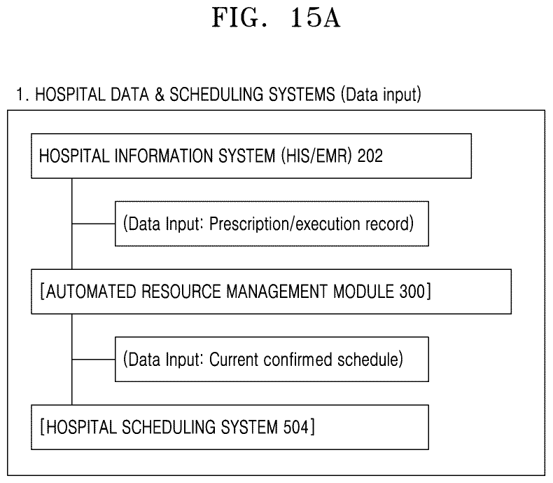

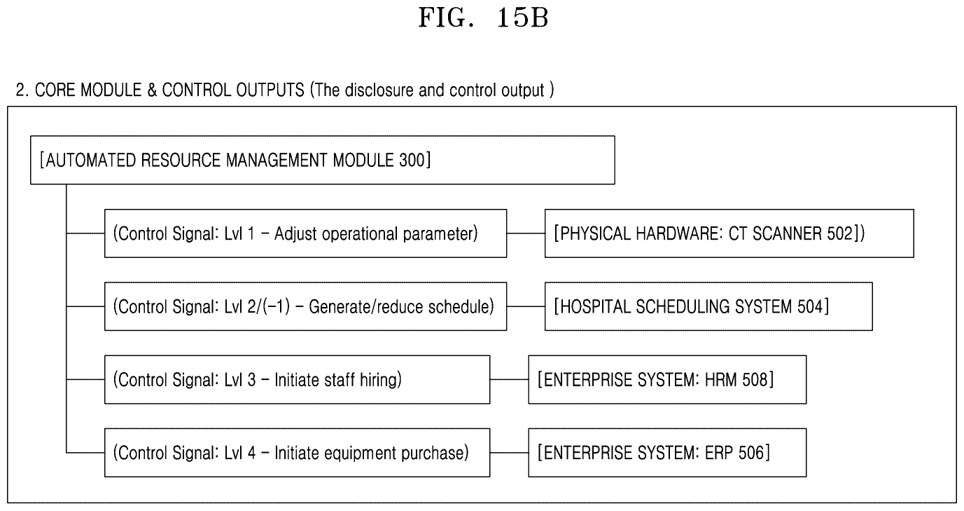

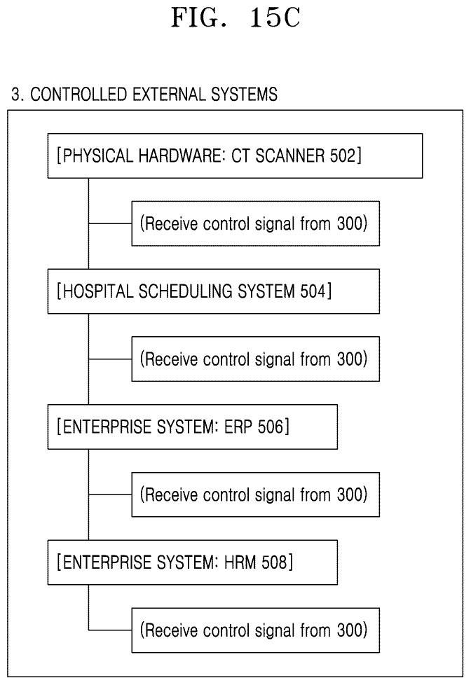

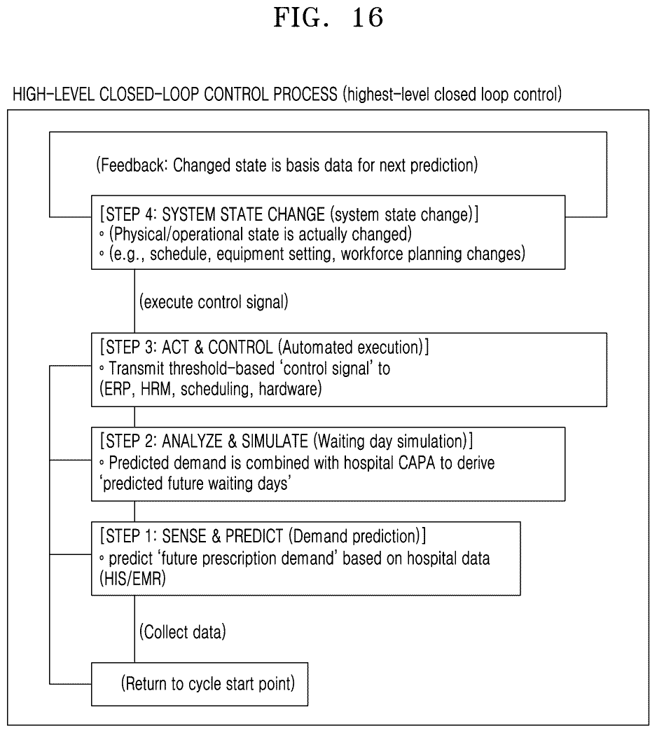

FIG. 14 is a diagram for describing a method of generating an automated control signal according to predicted waiting days, according to an embodiment of the disclosure. In addition, FIGS. 15A, 15B and 15C are diagrams for describing an integrated operation method with an external system and hardware of the disclosure. Also, FIG. 16 is a diagram for describing a cyclic operation method according to an embodiment of the disclosure.

Referring to FIG. 14, the core operation of the disclosure is illustrated, in which ‘predicted waiting days’, which is a simulation result, is received, and an automated control signal is transmitted to an external hospital system such as ERP or HRM according to a predefined threshold value.

Referring to FIGS. 15A, 15B and 15C, how the disclosure operates in integration with other core systems (HIS, ERP, HRM, etc.) and physical hardware (CT scanner) within a hospital infrastructure.

Referring to FIG. 16, the core cyclic operation principle of the disclosure, which proceeds from ‘prediction→simulation→automated control→feedback’, is illustrated.

The control logic of the system according to the disclosure consists of a rule-based engine that automatically executes a control action (system control action) mapped to a predefined threshold (trigger condition) when the predicted future (e.g., 6 months later) average waiting days satisfies the predefined threshold. This framework systematically manages hospital operations by dividing the same into several control levels.

For example, the control action according to the disclosure is divided into three categories of short-term, medium-term, and long-term depending on impact and execution time thereof, and is executed in conjunction with different hospital systems.

For example, direct hardware parameter adjustments may be made as short-term tactical control (on the order of weeks). For example, when the predicted waiting days reach a short-term threshold (e.g., greater than 40 days), the system directly accesses the reservation system database to dynamically reset the operating parameters of a specific CT scanner (physical hardware node). For example, it is possible to interface with a CT equipment management server or worklist server to send a control command to adjust a reservation slot interval (e.g., from 15 minutes in standard mode to 10 minutes in fast processing mode).

Specifically, by increasing a target throughput per hour, more inspections are performed in the same amount of time. For example, when the predicted waiting days suddenly exceed the threshold (over-demand), a resource control module interfaces with a worklist server to send a control command to reset a slot time interval parameter from ‘standard mode (15 minutes)’ to ‘fast processing mode (10 minutes)’. This is to technically minimize the patient turnaround time and idle time within the physical operating limits and safety margin of the equipment, thereby physically increasing the throughput per unit time. Conversely, when demand decreases and equipment overheating prevention or maintenance is required, the slot interval is extended to 20 minutes to automatically switch to ‘Eco Mode’, which reduces equipment load.

Additionally, the system continuously monitors changes in actual throughput and waiting days after changing the parameter, and when a control goal (e.g., reducing waiting days to less than 40 days) is not achieved, the system forms a feedback loop that automatically readjusts the parameters until the goal is reached. This is a typical characteristic of an automated control system.

For example, capacity augmentation may be achieved as a medium-term scheduling control (in units of several months). For example, when a higher medium-term threshold (e.g., greater than 60 days) is met, the system automatically generates an optimized weekend and overtime schedule table based on available staffing and equipment data and updates the table directly to the hospital's central scheduling system, making the same immediately actionable.

For example, capacity reduction may be implemented as a mid-term scheduling control (units of several months). For example, when predicted waiting days are very short (e.g., less than 15 days) and resource waste is expected, the system may intervene in the scheduling system to automatically reduce or eliminate previously set overtime or weekend work schedules, thereby optimizing operating costs.

For example, automated capital investment initiation (ERP integration) may be implemented as a long-term strategic control (six months or more). For example, when the system of the disclosure detects a long-term, structural capacity shortage (e.g., predicted waiting days exceeding 100 days), this is determined as a problem that cannot be resolved by simple operational measures. Here, the system generates a standardized data packet containing the technical specifications (e.g., contrast enhancement capability, processing speed) required for the introduction of a new CT scanner and transmits the same to the hospital's enterprise resource planning (ERP) system. This data packet acts as a trigger that automatically initiates a formal capital investment review and equipment purchase workflow within the ERP system.

The resource control module uses the Fast Healthcare Interoperability Resources (FHIR) protocol of the Health Level Seven (HL7) standard when communicating with external systems. Specifically, by generating a ‘DeviceRequest’ or ‘Appointment’ resource object structured in JSON format and sending the same to the target system (ERP, HRM) via RESTful API, an immediate process trigger is performed without manual input by a person.

For example, automated workforce planning (HRM integration) may be initiated as a long-term strategic control (6 months or more). For example, at similar long-term thresholds (e.g., greater than 80 days), the system generates a structured requisition specifying the job title needed (e.g., radiologist, nurse) and the number of positions to be filled, and sends the same to the human resources management (HRM) system. This serves to automatically initiate a formal new staff hiring process within the HRM system.

Traditional hospital management systems, even those with predictive capabilities, rely entirely on human managers to make final decisions and execute actions (e.g., generating purchase requisitions or posting job openings). However, according to the disclosure, these process initiation stages are automated, thereby eliminating humans from the decision-making process and allowing the data-based system's judgment to directly lead to strategic execution of an organization. This fundamentally changes the nature of hospital operations from periodic, human-centric processes to continuous, data-driven, automated processes. The ability to directly drive other enterprise computer systems based on such high confidence in the predicted results demonstrates that this technology is a sophisticated technological achievement that goes beyond simple information systems and autonomously manages core organizational functions.

TABLE 2
Automated Control Action Matrix by Predicted Waiting Day Threshold

| Trigger | | | | | |
|---|---|---|---|---|---|
| condition | | | | | |
| (predicted | | | | | |
| average | | | | | |
| waiting | | | System | | |
| days after | | Control | control | Nature of | Technical implementation |
| 6 months) | | level | action | improvement | method |
| >100 | days | 4 | Initiating | Long-term | Generate a structured data |
| | | | capital | hardware | packet containing technical |
| | | | investment | reconfiguration | specifications for the required |
| | | | process for | | equipment to initiate an |
| | | | acquisition of | | automated workflow in the |
| | | | new CT scanner | | hospital's asset |
| | | | equipment | | management/purchasing |
| | | | | | system (ERP). |
| >80 | days | 3 | Initiate a | Long-term | Automatically initiate a hiring |
| | | | human resources | operational | process by sending a |
| | | | process for | expansion | structured request specifying |
| | | | additional | | the job title and number of |
| | | | staff hiring | | persons needed to the human |
| | | | | | resources management |
| | | | | | system (HRM). |
| >60 | days | 2 | Generate and | Medium-term | Generate an optimized |
| | | | execute | capacity | weekend operating schedule |
| | | | weekend/ | expansion | table based on available |
| | | | overtime staffing | | staffing and equipment data, |
| | | | and equipment | | and update the table directly |
| | | | schedules | | into the hospital scheduling |
| | | | | | system. |
| >40 | days | 1 | Redeploying | Short-term | Physically reset the hourly |
| | | | staff on | operational | target throughput (operating |
| | | | weekday | optimization | parameter) of a specific CT |
| | | | shifts and | | scanner (hardware) by directly |
| | | | adjusting | | accessing the reservation |
| | | | hourly | | system database (DB). |
| | | | processing | | Automatic parameter |
| | | | targets for | | readjustment (feedback loop) |
| | | | specific | | by monitoring changes in |
| | | | network | | actual throughput and waiting |
| | | | nodes. | | days after changing the |
| | | | | | operating parameter when |
| | | | | | control targets are not met. |
| 20~30 | days | 0 | Maintain | Not | A monitoring system |
| | | | current | applicable | determines the current |
| | | | operating | | parameters as ‘optimal’. |
| | | | parameters | | Maintain current operating |
| | | | (optimal | | parameters in the system DB |
| | | | state) | | without sending a control |
| | | | | | action trigger. |
| <15 | days | −1 | Reduce or | Medium-term | Based on predicted low |
| | | | eliminate | capacity | demand data, update the |
| | | | weekend/ | reduction | hospital scheduling system to |
| | | | overtime staffing | | automatically reduce or delete |
| | | | and equipment | | existing weekend/overtime |
| | | | schedules. | | schedule tables. |
| <5 | days | −2 | Consolidation | Short-term | Direct access to the |
| | | | of weekday | operational | reservation system DB to |
| | | | shift work and | integration | shorten the operating time of a |
| | | | reduction of | | specific CT scanner (hardware) |
| | | | operating hours | | and adjust the target |
| | | | of certain | | throughput per hour (operating |
| | | | network nodes. | | parameter) downward. Reset |
| | | | | | parameters to integrate shift |
| | | | | | work by linking with the |
| | | | | | workforce scheduling system. |

The characteristic of the disclosure is not limited to any one of the three core technological elements (prediction engine, simulation system, and control framework), but lies in the synergistic effect of organically combining these to implement an unprecedented level of automated resource management system. While technologies in the related art have focused on addressing fragmented issues such as demand forecasting and real-time patient flow management, the disclosure is fundamentally different in that the entire process from forecasting to strategic execution is completed by a single, integrated, closed-loop system. Real-time time series cross-validation and parameter tuning on these thousands of data sets is beyond human mental capacity and may only be performed using high-performance computer systems.

The system of the disclosure overcomes the limitations of existing technologies through the following specific technical differences. For the classical problem of time series forecasting, a tabular regression approach is applied, which competitively evaluates a large pool of regression models and combines the results into a soft voting ensemble. This is a systematic methodology for achieving high accuracy optimized for specific domain data, and is a core foundation technology that ensures the reliability of the entire automated system. Additionally, a ‘digital twin’ is implemented, which simulates the complex realities of hospital operations with high fidelity through dynamic CAPA modeling, a ‘waterfall’ reallocation algorithm that applies differential rules by department, and realistic waiting days calculation logic. The sophistication of these simulations plays a crucial role in transforming abstract predictions into reliable, concrete actionable evidence. Moreover, the greatest technological leap of the present system is the ability to exercise autonomous control over dramatically different time scales, from short-term control such as real-time hardware parameter tuning to long-term capital investment and workforce planning initiatives through ERP/HRM system integration. This indicates that the system has evolved beyond the role of providing information provided by existing management tools to the level of directly executing strategic decisions of the organization.

In conclusion, the disclosure provides a concrete and practical technical solution for solving the problem of hospital resource management. The present system fundamentally improves the operational method of the technology infrastructure of the hospital by directly controlling the operation of physical hardware (CT scanners) and initiating automated processes within other enterprise computer systems (ERP, HRM, scheduling systems). This goes beyond abstract concepts that simply organize human activity or suggest business methods, and demonstrates that computer systems act as a tangible technological unit of improving hospital operations, reducing costs, and ultimately improving the quality of patient care.

The apparatus and/or system described above may be implemented as a hardware component, a software component, and/or a combination of hardware components and software components. Apparatuses and components described in the embodiments may be implemented using one or more general-purpose or special-purpose computers, for example, a processor, a controller, an arithmetic logic unit (ALU), a digital signal processor, a microcomputer, a field programmable gate array (FPGA), and a programmable logic unit (PLU), a microprocessor, or any other device capable of executing and responding to instructions. A processing device may run an operating system (OS) and one or more software applications running on the operating system. A processing device may also access, store, manipulate, process, and generate data in response to execution of software. For convenience of understanding, there are cases in which one processing device is used, but it will be obvious to those skilled in the art that a processing device may include a plurality of processing elements and/or a plurality of types of processing elements. For example, a processing device may include a plurality of processors or a single processor and a controller. Other processing configurations such as parallel processors are also possible.

Software may include computer program, code, instructions, or a combination of one or more of these, and may configure a processing device or independently or collectively configure give a command to the processing device to operate as desired. Software and/or data may be permanently or temporarily embodied in any tangible machine, a component, a physical device, virtual equipment, a computer storage medium or device, or in a transmitted signal wave in order to be interpreted by or provide instructions or data to a processing device. The software may be distributed on networked computer systems and stored or executed in a distributed manner. Software and data may be stored on one or more computer-readable recording media.

The method according to an embodiment may be embodied as program commands executable by various computer means and may be recorded on a computer-readable recording medium. The computer-readable recording medium may include program commands, data files, data structures, and the like separately or in combinations. The program commands recorded on the computer-readable recording medium may be specially designed and configured for the embodiments or may be well-known and be available to those of ordinary skill in the art of computer software. Examples of the computer-readable recording medium include magnetic media such as a hard disk, a floppy disk, or a magnetic tape, optical media such as a CD-ROM or a DVD, magneto-optical media such as a floptical disk, and a hardware device specially configured to store and execute program commands such as a ROM, a RAM, or a flash memory. Examples of the program commands include advanced language codes that may be executed by a computer by using an interpreter or the like as well as machine language codes made by a compiler. The hardware devices described above may be configured to operate as one or more software modules to perform the operations of the embodiments, and vice versa.

According to an embodiment as described above, a method for predicting waiting days for an examination that requires a reservation at a hospital, an predictive automated medical examination resource management system, and a computer program stored in a recording medium to execute the method may be implemented. The scope of the disclosure, however, is not limited by these effects.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A system for performing prediction-based automated medical examination resource management, the system comprising:
a memory storing instructions; and
at least one processor configured to execute the instructions to implement:
a communication module configured to collect raw data related to computerized tomography (CT) examinations from an electronic medical record (EMR) system or a hospital information system (HIS);
a predictive prescription forecasting engine configured to predict a predicted demand curve representing a trend of a future average number of daily prescriptions based on the raw data by using machine learning;
a dynamic waiting days simulation module configured to derive a predicted waiting day curve indicating expected waiting days for each future date by using the predicted demand curve and available examination capacity (CAPA) data of a CT scanner equipment for each date; and
a resource control module configured to automatically transmit a control signal to a hospital scheduling system or CT scanner equipment when the expected waiting date for each future date on the predicted waiting day curve satisfies a predefined threshold condition,
wherein the communication module, the predictive prescription forecasting engine, the dynamic waiting day simulation module, and the resource control module interact with each other in a closed loop structure to automatically adjust hospital examination resources.

2. The system of claim 1, wherein the predictive prescription forecasting engine is configured to learn by dividing the raw data by department and setting time series elements as independent variables and the average number of daily prescriptions as dependent variables.

3. The system of claim 2, wherein the predictive prescription forecasting engine is configured to select a preset number of a plurality of machine learning models based on a mean absolute error (MAE) from among pre-stored evaluation target models, and to combine prediction results of the plurality of selected machine learning models.

4. The system of claim 3, wherein the predictive prescription forecasting engine is configured to generate a single prediction value by combining the prediction results through a soft voting method that weights and averages prediction probability values of the plurality of selected machine learning models.

5. The system of claim 1, wherein the dynamic waiting day simulation module is configured to probabilistically distribute an initial reservation demand to future dates according to a lag distribution based on a prescription-to-execution lag distribution representing a probability distribution of a period of time required from a date a CT prescription is issued until the actual examination is performed and based on the predicted demand curve.

6. The system of claim 5, wherein the dynamic waiting day simulation module is configured to detect overbooking by comparing the total reservation demand allocated for each date with an available examination capacity for each future date.

7. The system of claim 6, wherein the dynamic waiting day simulation module is configured to process overbooking based on a waterfall method that reallocates overbooking to a next available date according to a rule preset for each department when overbooking is detected.

8. The system of claim 7, wherein the dynamic waiting day simulation module is configured to, when the overbooking is not detected, calculate, as expected waiting days for each future date, a period until the first day of two consecutive weekdays when the number of reservations to the capacity for each future date is equal to or less than a predetermined ratio.

9. The system of claim 1, wherein the resource control module is configured to, when the expected waiting days for each future date exceed a predefined threshold, transmit a control signal for adjusting physical operating parameters of a CT scanner or a control signal for automatically changing a work schedule of a hospital scheduling system, and the control signal resets a slot time interval parameter from a standard mode to a fast processing mode.

10. The system of claim 1, wherein the resource control module is configured to, when the expected waiting days for each future date exceed a predefined medium—to long-term reference threshold, automatically generate a new equipment acquisition workflow in an enterprise resource planning (ERP) system or to send a control signal to automatically initiate a staff hiring process in a human resources management (HRM) system.
